(19) United States
(12) Patent Application Publication
(10) Pub. No.: US 2007/0100383 A1
(43) Pub. Date: May 3, 2007

(54) Lead and delivery system to detect and treat a myocardial infarction region

(75) Inventors: Joseph M. Pastore, Woodbury, MN (US); Rodney W. Salo, Fridley, MN (US); Ronald W. Heil JR., Roseville, MN (US); Randy Westlund, River Falls, WI (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 11/261,202

(22) Filed: Oct. 28, 2005

(51) Int. Cl. A61N 1/36

(52) U.S. Cl. 607/9; 607/129

(57) ABSTRACT

A method includes mounting an anchor member to a surface of a heart, the anchor member having a tension member coupled to the anchor member, advancing a lead body along the tension member, the lead body including a plurality of electrodes disposed along the lead body, identifying an MI region of the heart, positioning the plurality of electrodes at or near the MI region, affixing the tension member to the lead body to hold the electrodes in position, and delivering pulses through the plurality of electrodes to the heart.

### FIELD

This invention pertains to methods of treating cardiac disease and cardiac rhythm management devices such as pacemakers and other implantable devices.

### BACKGROUND

A myocardial infarction (MI) is the irreversible damage done to a segment of heart muscle by ischemia, where the myocardium is deprived of adequate oxygen and metabolite removal due to an interruption in blood supply. It is usually due to a sudden thrombotic occlusion of a coronary artery, commonly called a heart attack. If the coronary artery becomes completely occluded and there is poor collateral blood flow to the affected area, a transmural or full-wall thickness infarct can result in which much of the contractile function of the area is lost. Over a period of one to two months, the necrotic tissue heals, leaving a scar. The most extreme example of this is a ventricular aneurysm where all of the muscle fibers in the area are destroyed and replaced by fibrous scar tissue.

Left ventricular remodeling is a physiological process in response to the hemodynamic effects of the infarct that causes changes in the shape and size of the left ventricle. Remodeling is initiated in response to a redistribution of cardiac stress and strain caused by the impairment of contractile function in the infarcted area as well as in nearby and/or interspersed viable myocardial tissue with lessened contractility due to the infarct. The remodeling process following a transmural infarction starts with an acute phase which lasts only for a few hours. The infarcted area at this stage includes tissue undergoing ischemic necrosis and is surrounded by normal myocardium. Over the next few days and months after scar tissue has formed, global remodeling and chamber enlargement occur in a third phase due to complex alterations in the architecture of the left ventricle involving both infarcted and non-infarcted areas. Remodeling is thought to be the result of a complex interplay of hemodynamic, neural, and hormonal factors.

As described above, the remodeling process begins immediately after a myocardial infarction. Until scar tissue forms, the infarcted area is particularly vulnerable to the distending forces within the ventricle and undergoes expansion over a period of hours to days as shown in a second phase of remodeling. Preventing or minimizing such post-infarct remodeling is a concern.

### SUMMARY

In one aspect, a method includes mounting an anchor member at a surface of a heart, the anchor member having a tension member coupled to the anchor member. The method further includes advancing a lead body along the tension member, the lead body including a plurality of electrodes disposed along the lead body. The method includes identifying an MI region of the heart, positioning the plurality of electrodes at or near the MI region, affixing the tension member to the lead body to hold the electrodes in position, and delivering pulses through the plurality of electrodes to the MI region.

### BRIEF DESCRIPTION OF THE DRAWINGS

FIG. 1 is a system diagram of a pulse generator device according to one embodiment.

FIG. 2 shows a system diagram of a sensing system according to one embodiment.

FIG. 3 shows a view of a lead according to one embodiment.

FIG. 4 shows a tool for implanting a lead, in accordance with one embodiment.

FIG. 5 shows the lead of FIG. 3 implanted in the heart.

FIG. 6 illustrates a method according to one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

The degree to which a heart muscle fiber is stretched before it contracts is termed the preload, while the degree of tension or stress on a heart muscle fiber as it contracts is termed the afterload. The maximum tension and velocity of shortening of a muscle fiber increases with increasing preload, and the increase in contractile response of the heart with increasing preload is known as the Frank-Starling principle. When a myocardial region contracts late relative to other regions, the contraction of those other regions stretches the later contracting region and increases its preloading, thus causing an increase in the contractile force generated by the region. Conversely, a myocardial region that contracts earlier relative to other regions experiences decreased preloading and generates less contractile force. Because pressure within the ventricles rises rapidly from a diastolic to a systolic value as blood is pumped out into the aorta and pulmonary arteries, the parts of the ventricles that contract earlier during systole do so against a lower afterload than do parts of the ventricles contracting later. Thus, if a ventricular region can be made to contract earlier than parts of the ventricle, it will be subjected to both a decreased preload and afterload which decreases the mechanical stress experienced by the region relative to other regions during systolic contraction. The region will also do less work thus lessening its metabolic demands and the degree of any ischemia that may be present.

If the region at and around an infarct were made to contract during early systole, it would be subjected to less distending forces and less likely to undergo expansion, especially during the period immediately after a myocardial infarction. In order to cause early contraction and lessened stress, electrostimulatory pacing pulses may be delivered to one or more sites in or around the infarct in a manner that pre-excites those sites relative to the rest of the ventricle. (As the term is used herein, a pacing pulse is any electrical stimulation of the heart of sufficient energy to initiate a propagating depolarization, whether or not intended to enforce a particular heart rate.)

In a normal heartbeat, the specialized His-Purkinje conduction network of the heart rapidly conducts excitatory impulses from the sino-atrial node to the atrio-ventricular node, and thence to the ventricular myocardium to result in a coordinated contraction of both ventricles. Artificial pacing with an electrode fixed into an area of the myocardium does not take advantage of the heart's normal specialized conduction system for conducting excitation throughout the ventricles because the specialized conduction system can only be entered by impulses emanating from the atrio-ventricular node. Thus the spread of excitation from a ventricular pacing site must proceed only via the much slower conducting ventricular muscle fibers, resulting in the part of the ventricular myocardium stimulated by the pacing electrode contracting well before parts of the ventricle located more distally to the electrode. This pre-excitation of a paced site relative to other sites can be used to deliberately change the distribution of wall stress experienced by the ventricle during the cardiac pumping cycle.

Pre-excitation of the infarct region relative to other regions unloads the infarct region from mechanical stress by decreasing its afterload and preload, thus preventing or minimizing the remodeling that would otherwise occur. In addition, because the contractility of the infarct region is impaired, pre-excitation of the region may result in a resynchronized ventricular contraction that is hemodynamically more effective. Decreasing the wall stress of the infarct region may also lessen its oxygen requirements and lessens the probability of an arrhythmia arising in the region.

Pacing therapy to unload the infarct region may be implemented by pacing the ventricles at a single site in proximity to the infarct region or by pacing at multiple ventricular sites in such proximity. In the latter case, the pacing pulses may be delivered to the multiple sites simultaneously or in a defined pulse output sequence. As described below, the single-site or multiple site pacing may be performed in accordance with a bradycardia pacing algorithm such as an inhibited demand mode or a triggered mode.

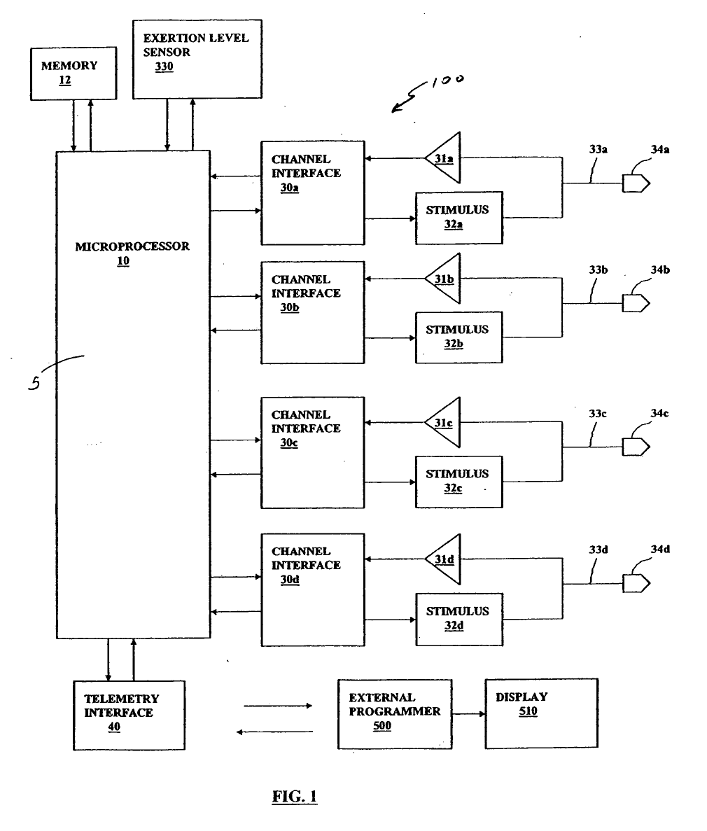

FIG. 1 shows a system diagram of a pulse generator 100, according to one embodiment. Pulse generator 100 includes multiple sensing and pacing channels which may be physically configured to sense and/or pace multiple sites in the atria or the ventricles. Pulse generators are usually implanted subcutaneously in the patient's chest and connected to sensing/pacing electrodes by leads either threaded through the vessels of the upper venous system to the heart or by leads that penetrate the chest wall. (As the term is used herein, a “pulse generator” should be taken to mean any cardiac rhythm management device with a pacing functionality regardless of any other functions it may perform.)

A controller 5 of the pulse generator 100 includes a microprocessor 10 which communicates with a memory 12 via a bidirectional data bus. The controller could be implemented by other types of logic circuitry (e.g., discrete components or programmable logic arrays) using a state machine type of design, but a microprocessor-based system is preferable. As used herein, the term “circuitry” should be taken to refer to either discrete logic circuitry or to the programming of a microprocessor. The memory 12 typically comprises a ROM (read-only memory) for program storage and a RAM (random-access memory) for data storage. Shown in the figure are four exemplary sensing and pacing channels designated “a” through “d” comprising electrodes 34a-d, leads 33a-d, sensing amplifiers 31a-d, pulse energy generators 32a-d, and channel interfaces 30a-d.

Although only one electrode for each lead is shown in the figure, in some embodiments the leads may be either unipolar leads, where a single electrode referenced to the device housing is used for sensing and pacing, or bipolar leads which include two closely spaced electrodes for sensing and pacing. Moreover, in some embodiments, a single lead can include 3, 4, 5, or more electrodes, with each electrode independently coupled to controller 5.

The channel interfaces 30a-d communicate bidirectionally with microprocessor 10, and each interface can include analog-to-digital converters for digitizing sensing signal inputs from the sensing amplifiers and registers that can be written to by the microprocessor in order to output pacing pulses, change the pacing pulse amplitude, and adjust the gain and threshold values for the sensing amplifiers. An exertion level sensor 330 (e.g., an accelerometer, a minute ventilation sensor, or other sensor that measures a parameter related to metabolic demand) enables the controller to adapt the pacing rate in accordance with changes in the patient's physical activity. In some embodiments, a telemetry interface 40 is also provided for communicating with an external programmer 500 which has an associated display 510.

In certain patients, pacing of sites in proximity to an infarct or within ischemic regions may be less excitable than normal and require an increased pacing energy in order to achieve capture (i.e., initiating of a propagating action potential). For each channel, the same electrode pair can be used for both sensing and pacing. In this embodiment, bipolar leads that include two electrodes are used for outputting a pacing pulse and/or sensing intrinsic activity. Other embodiments may employ a single electrode for sensing and pacing in each channel, known as a unipolar lead.

The controller 5 controls the overall operation of the device in accordance with programmed instructions stored in memory 12, including controlling the delivery of paces via the pacing channels, interpreting sense signals received from the sensing channels, and implementing timers for defining escape intervals and sensory refractory periods. The sensing circuitry of the pacemaker detects a chamber sense, either an atrial sense or ventricular sense, when an electrogram signal (i.e., a voltage sensed by an electrode representing cardiac electrical activity) generated by a particular channel exceeds a specified detection threshold. Pacing algorithms used in particular pacing modes employ such senses to trigger or inhibit pacing, and the intrinsic atrial and/or ventricular rates can be detected by measuring the time intervals between atrial and ventricular senses, respectively.

The controller 5 is capable of operating the device in a number of programmed pacing modes which define how pulses are output in response to sensed events and expiration of time intervals. Most pacemakers for treating bradycardia are programmed to operate synchronously in a so-called demand mode where sensed cardiac events occurring within a defined interval either trigger or inhibit a pacing pulse. Inhibited demand pacing modes utilize escape intervals to control pacing in accordance with sensed intrinsic activity such that a pacing pulse is delivered to a heart chamber during a cardiac cycle only after expiration of a defined escape interval during which no intrinsic beat by the chamber is detected. Escape intervals for ventricular pacing can be restarted by ventricular or atrial events, the latter allowing the pacing to track intrinsic atrial beats. Multiple excitatory stimulation pulses can also be delivered to multiple sites during a cardiac cycle in order to both pace the heart in accordance with a bradycardia mode and provide regional unloading of the myocardium to reduce regional stress and attenuate remodeling.

Pulse generator 100 can be configured such that multiple cardiac sites are sensed and/or paced. As described below, this allows those sites to be monitored to determine if any are infarcted. Once one or more such sites are identified, the device may be programmed to initiate remodeling reduction pacing that pre-excites the MI region or sites. Initiation of remodeling reduction pacing may involve altering the device's pulse output configuration and/or sequence, where the pulse output configuration specifies a specific subset of the available electrodes to be used for delivering pacing pulses and the pulse output sequence specifies the timing relations between the pulses.

In the case where the pre-excitation pacing of a ventricle is delivered at multiple sites, the sites may be paced simultaneously or in accordance with a particular pulse output sequence that specifies the order in which the sites are to be paced during a single beat. As discussed above, one benefit of pre-excitation pacing is that pacing unloads the peri-MI region and MI region while minimally compromising hemodynamic function. Another possible benefit of pre-excitation pacing of the infarct region may be resynchronization of the contraction that results in hemodynamic improvement. In either case, the therapy may be more successful if multiple ventricular sites are paced in a specified sequence such that certain of the pacing sites are pre-excited earlier than others during a single beat. Pre-excitation pacing may involve biventricular pacing with the paces to right and left ventricles delivered either simultaneously or sequentially, with the interval between the paces termed the biventricular offset (BVO) interval (also sometimes referred to as the LV offset (LVO) interval or VV delay). The offset interval may be zero in order to pace both ventricles simultaneously, or non-zero in order to pace the left and right ventricles sequentially. As the term is used herein, a negative BVO refers to pacing the left ventricle before the right, while a positive BVO refers to pacing the right ventricle first.

In atrial tracking and AV sequential pacing modes, another ventricular escape interval is defined between atrial and ventricular events, referred to as the AV delay (AVD) interval, where a ventricular pacing pulse is delivered upon expiration of the AV delay interval if no ventricular sense occurs before. In an atrial tracking mode, the atrio-ventricular pacing delay interval is triggered by an atrial sense and stopped by a ventricular sense or pace. An atrial escape interval can also be defined for pacing the atria either alone or in addition to pacing the ventricles. In an AV sequential pacing mode, the atrio-ventricular delay interval is triggered by an atrial pace and stopped by a ventricular sense or pace. Atrial tracking and AV sequential pacing are commonly combined so that an AVD interval starts with either an atrial pace or sense. As the term is used herein for biventricular pacing, the AVD interval refers to the interval between an atrial event (i.e., a pace or sense in one of the atria, usually the right atrium) and the first ventricular pace which pre-excites one of the ventricles, and the pacing instant for the non-pre-excited ventricle is specified by the BVO interval so that it is paced at an interval AVD+BVO after the atrial event. With either biventricular or left ventricle-only pacing, the AVD interval may be the same or different depending upon whether it is initiated by an atrial sense or pace (i.e., in atrial tracking and AV sequential pacing modes, respectively). A common way of implementing biventricular pacing or left ventricle-only pacing is to base the timing upon only right ventricular activity so that ventricular escape intervals are reset or stopped by right ventricular senses.

In order to place one or more pacing electrodes in proximity to an MI region, the area of the infarct can be identified by assessment of myocardial wall stress, for example. In order to assess local myocardial wall stress, the action potential duration during systole, also referred to herein as the activation-recovery interval, can be measured by pulse generator 100 (or other controller coupled to sensing electrodes) at those sites where sensing electrodes are disposed. Because the bipolar electrodes “see” a smaller volume of the myocardium, it may be desirable to use bipolar sensing electrodes rather than unipolar electrodes for measuring the activation-recovery interval at the electrode sites. In one implementation, the controller is programmed to measure the activation-recovery interval as the time between a detected depolarization and a detected repolarization in an electrogram generated by a sensing channel. Sensing channels can be designed to detect both depolarizations (i.e., conventional atrial or ventricular senses) and repolarizations.

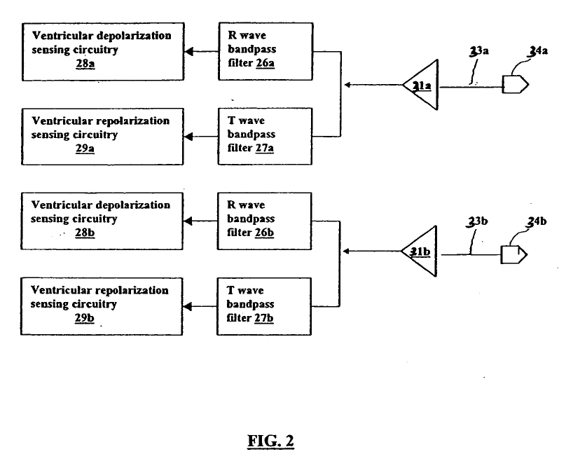

FIG. 2 illustrates how this may be implemented in a ventricular sensing channel, in accordance with one embodiment. When the channel is awaiting a ventricular sense, the electrogram signal is passed through an R wave bandpass filter (26a or 26b) with passband characteristics selected to match the frequency content of a ventricular depolarization. The ventricular depolarization sensing circuitry (28a or 28b) then compares the filtered electrogram signal with a threshold to detect when a ventricular sense occurs. After a ventricular sense occurs, the channel awaits a ventricular repolarization during a specified time frame (e.g., between 50 and 500 milliseconds after the ventricular depolarization). During this time, the electrogram signal is passed through a T wave bandpass filter (27a or 27b) that has a passband characteristic conforming to the frequency content of a ventricular repolarization which is generally lower than that of a ventricular depolarization. The ventricular repolarization sensing circuitry (29a or 29b) then compares the filtered electrogram signal with a threshold to determine when the repolarization occurs. The channel may continue to monitor for depolarizations during this time in case the repolarization is undersensed. A similar scheme with atrial depolarization and repolarization bandpass filters and sensing circuits may be implemented to detect atrial repolarizations.

The bandpass filters may be implemented as analog filters that operate directly on the electrogram signal received from the electrodes or may be switched capacitor-type filters that sample the electrogram signal into a discrete-time signal which is then filtered. Alternatively, the electrogram signal can be sampled and digitized by an A/D converter in the channel interface with the bandpass filtering implemented in the digital domain by a dedicated processor or code executed by the controller.

After measuring the activation-recovery interval at a plurality of myocardial sites, sites that are infarcted may be identified with a specified threshold criterion applied to the activation-recovery interval. That is, a site is identified as infarcted when its measured activation-recovery interval is below the specified threshold value. Because the cardiac action potential normally varies with heart rate, it may be desirable to measure activation-recovery intervals during intrinsic beats for the purpose of assessing myocardial remodeling only when the heart rate is within a specified range. Activation-recovery intervals can also be measured during paced beats while pacing pulses are delivered at a specified rate. In the case of a paced beat, the depolarization corresponds to an evoked response detected by the sensing channel, while the repolarization is similar to an intrinsic beat. Alternatively, the threshold criterion for assessing the myocardial wall based upon the activation-recovery interval may be adjusted in accordance with the measured intrinsic heart rate or pacing rate.

Another technique that can be used to identify infarcted sites is the phenomena of mechanical alternans. When oscillations in pulse pressure are detected in a patient, referred to as pulsus alternans it is generally interpreted by clinicians as a sign of left ventricular dysfunction. Localized alternations in local wall stress, as revealed by alternations in the activation-recovery interval, may similarly indicate that the site is an MI region. MI sites may therefore be identified by detecting oscillations in the measured activation-recovery interval either instead of, or in addition to, the threshold criterion for the activation-recovery interval discussed above.

In one example, identifying an MI region can include sensing a decrease in R wave amplitude via the sensing electrodes. For example, sensing of an MI region can be detected by decrease in R wave amplitude as a lead is maneuvered in the heart or by comparing R wave amplitudes between the different electrodes on the lead.

Once the MI region is identified, the information may be communicated to an external programmer via a telemetry link and used by a clinician in planning further treatment. A wall remodeling parameter for each electrode site may be determined from the length of the activation-recovery interval as well as a parameter representing the average overall myocardial wall stress. As described below, the device may also be programmed to alter its pacing mode so as to mechanically unload the MI region

The MI region can be mechanically unloaded during systole by delivering one or more pacing pulses in a manner such that the MI region is pre-excited relative to other regions of the myocardium. Such pacing subjects the MI region to a lessened preload and afterload during systole, thus reducing the wall stress. By unloading a myocardial region in this way over a period of time, reversal of undesirable myocardial remodeling may also be effected.

Pacing for myocardial wall remodeling reduction may be delivered in accordance with a programmed bradycardia pacing mode and thus also provide therapy for bradycardia as well. Such pacing also may or may not include multi-site pacing for purpose of also providing cardiac resynchronization therapy. What affects localized remodeling reduction is the pre-excitation of one or more myocardial regions relative to other regions during systole. This may be accomplished in certain situations with single-site pacing and in others with multi-site resynchronization pacing that also can improve the pumping function of the heart. In the latter case, the pacing pulse output configuration and sequence that produces optimum resynchronization may or may not also deliver optimum therapy for reduction of myocardial wall stress.

In one embodiment, pulse generator 100 can be configured with a plurality of pacing/sensing electrodes disposed in both ventricles at selected sites. The device is programmed to normally deliver pacing pulses to one or more selected pacing electrodes, referred to as a pulse output configuration, and in a specified time sequence, referred to as a pulse output sequence. One such site then is identified as a MI region by measurement of activation-recovery intervals at the electrodes during either intrinsic or paced beats, or some other means, such as R wave amplitude, and the device is programmed by initiate remodeling reduction pacing for that site.

In one example, the device normally delivers bradycardia pacing at a single ventricular site, and then switches the pacing configuration to deliver pacing pulses to the stressed site. Single-site pacing that pre-excites the ventricle at this site results in the MI region being excited before other regions of the ventricular myocardium as the wave of excitation spreads from the paced site. In order to reduce remodeling at the identified site, the pulse output configuration is modified, if necessary, to include the MI region, and the pulse output sequence is selected such that the MI region and/or peri-MI region is excited before other regions as the wave of excitation spreads from the multiple pacing sites.

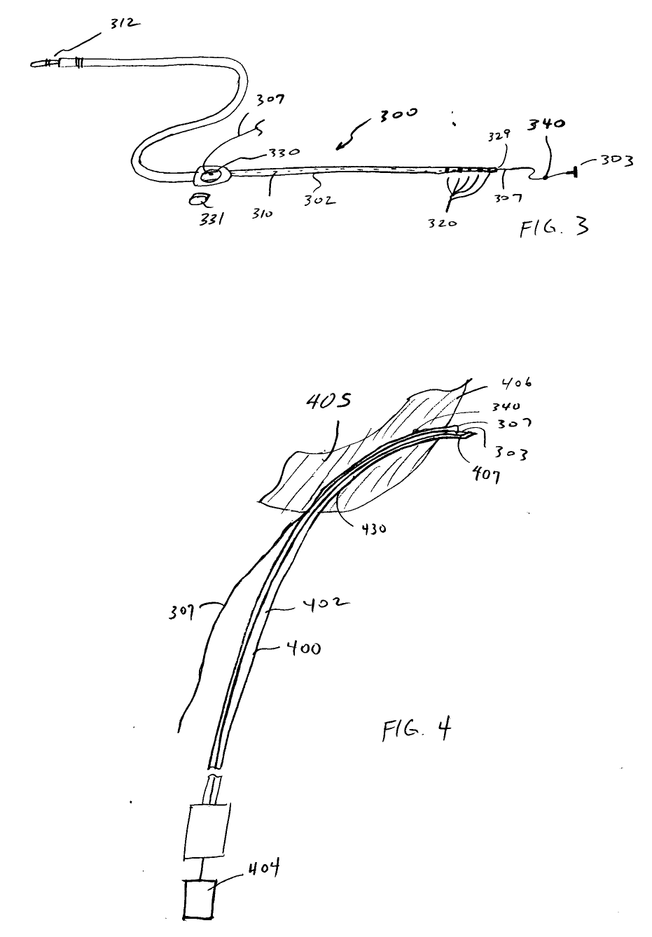

FIG. 3 shows a lead 300 according to one embodiment. Lead 300 includes four or five (or more) electrodes 320 designed to be implanted in an MI region and/or a peri-MI region. A proximal end of the lead includes a terminal 312 to connect to a pulse generator, such as a pulse generator discussed above. In this example lead 300 includes a lead body 302 having electrodes 320 disposed on its distal end. A passage 310 through at least a portion of the lead body accepts therein a flexible tension member 307, such as a thread. Tension member 307 extends through a distal opening 329 at the end of the lead and is coupled to an anchor member 303, such as a T-bar. Lead body 302 can translate along the tension member 307 via passage 310 and, as will be discussed below, lead 300 is configured such that electrodes 320 can be fixed in an operating position in cardiac muscle tissue (myocardium) with the aid of anchor member 303 and tension member 307.

At a distance from its end and from the anchor member 303, lead body 302 includes an exit opening 330 from passage 310 for the tension member 307 to exit through. Tension member 307 can be fixed at or outside this opening by a knot or in some other manner. In addition, after the knotting, the exit opening 330 can be closed with a medical adhesive, for example, to improve fixation of the knot. In another example, to address this closure issue, a relatively soft silicone rubber plug 331 or wedge component can be inserted into the opening 330. In this case, however, the opening 330 can include a restraining lip feature. For example, in use, after anchor member 303 is anchored to the heart and lead 300 is correctly positioned via tension member 307, a knot in tension member 307 can be prepared to prevent the lead from moving. The soft wedge or plug 331 would then be compressed and inserted through the opening 330. Upon expansion inside the opening, the plug 331 would cover and seal the knot in place. Also, a small amount of medical adhesive can be applied to an inner surface of plug 331 prior to inserting the plug. In this fashion, the uncured medical adhesive would be buried within the lead body and would never be exposed to tissue. Thus, with the anchor member 303 located at a surface of the myocardium and tension member 307 fixed at opening 330, lead 300 is prevented from moving forward or backward.

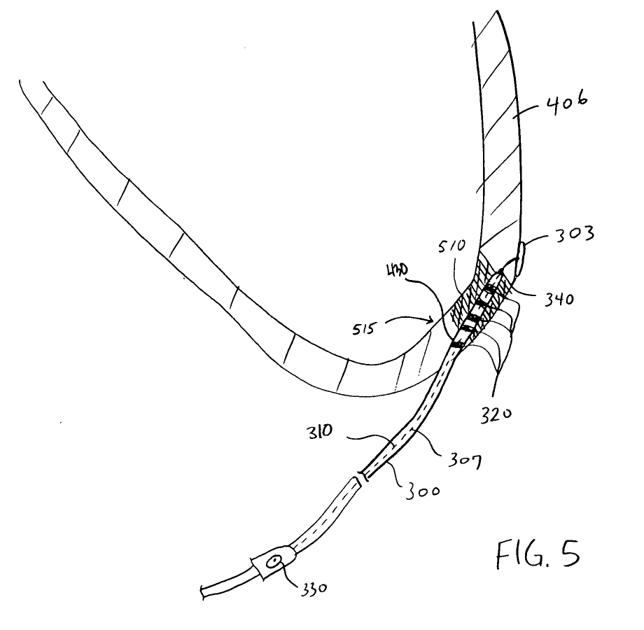

FIG. 4 shows a view of a tool 400 used to insert anchor member 303 in or on heart 405. To position and fix the anchor member 303 on or in the myocardium 406, tool 400 includes a thin, flexible stylet 404. Anchor member 303 can include a hole in one end of the anchor member to receive the stylet therein. The tool 400 also includes a cannula 402. In this example, cannula 402 is a rigid, hollow, curved member used to puncture or prick a canal 430 in or through the myocardium 406, through which channel the anchor member 303 is pushed into its operating position at the outside of the heart using the stylet 404.

Anchor member 303, along with stylet 404 gripping it, is guided in cannula 402. The tension member 307 fastened to the anchor member 303 exits cannula 402 through a hole 407 located near an end of cannula 402 and runs along the outside of the cannula 402. When the anchor member 303, guided by the stylet 404, leaves the myocardial canal 430, the stylet 404 is withdrawn from the hole in the end of the anchor member. The anchor member 303 then swings out into its operating position. For example, the anchor member 303 can include a rod-like shape, wherein the hole runs in the longitudinal direction of the anchor member 303 and has the form of a blind hole. The tension member 307 can be attached approximately at the center between both ends of the anchor member 303, extending transverse with respect to the orientation of the anchor member 303. The anchor member 303 can thereby rest lightly against the exterior surface of the heart, transversely to the myocardial canal 403, and there anchor the tension member 307.

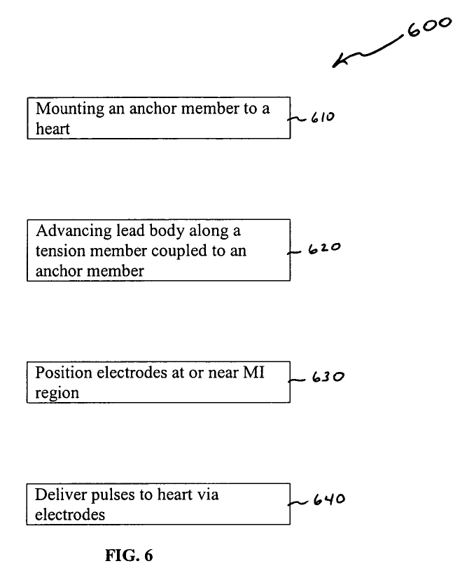

Referring now also to FIG. 5, after the creation of the myocardial canal 430, the cannula 402 is withdrawn and the lead 300 is pushed into the myocardial canal 430 along passage 310 via the tension member 307 that still runs through the myocardial canal 430 and is secured by the anchor member 303, until the front end of the lead impinges on a stop 340, such as a knot, and comes to rest in the myocardium or, with the electrodes in the myocardium. Using this stop 340, the end of the lead 300 can be positioned in the heart in the operating position at a fixed distance from the anchor member 303. The stop 340 can include a simple knot on the tension member 307 or some other thickening or projection or cross-sectional enlargement on the tension member 307, with the cross-section of the stop 340 exceeding the inner cross section of the guide passage 310 or a narrowed section of the guide passage. Using this stop 340, which is impinged on by the front end of the lead, and the knotting of the tension member 307 at the exit opening 330 at the rear part of the lead, the tension member 307 is made taut between these two fastening points, thereby holding lead 300 and electrodes 320 guided thereon in their operating position. Subsequently, the tension member 307 is affixed to the exit opening 330 and thereby the lead 300 is fastened at its back end to the tension member 307. The lead can be implanted such that electrodes 320 are positioned in the MI region 510 or a peri-MI region 515.

In some embodiments, anchor member 303 can include variously-configured collapsible parts or elements or pins or wings, which are folded down against a spring force during insertion with the aid of the stylet 404 and the cannula 402 and which after leaving the cannula 402, or, as the case may be, the myocardial canal 430, spread out or unfold or swing out by virtue of the restoring force and assume a position transverse to the tension member 307.

Thus, in using the lead 300, a method such as shown in FIG. 6 can be used. Method 600 includes: mounting an anchor member to a surface of a heart (610); advancing a lead body along the tension member (620); positioning the plurality of electrodes at or near the MI region (630); and delivering pulses through the plurality of electrodes to the heart (640).

As discussed above, identifying the MI region can include sensing a decrease in R wave amplitude or measuring activation-recovery intervals at a plurality of myocardial sites.

Mounting the anchor member can include placing the anchor through an incision on the thorax and mounting it to the epidcardium, for example.

In the examples described above, the device is programmed to alter its pacing mode when a MI region is identified by modifying the pulse output configuration and/or sequence to pre-excite the stressed site. Remodeling reduction pacing may be augmented where the pacing pulses are delivered in a demand mode by decreasing the escape interval used to pace the MI region (e.g., the ventricular escape interval or the AV delay interval in the case of dual-chamber pacing). In another example, the device is configured with multiple sensing/pacing electrodes but is programmed to deliver neither bradycardia nor pre-excitation pacing during normal operation. After a MI region is identified, a pacing mode is initiated such that the MI region and/or peri-MI region is pre-excited in a timed relation to a triggering event that indicates an intrinsic beat has either occurred or is imminent such as immediately following the earliest detection of intrinsic activation elsewhere in the ventricle. Such activation may be detected from an electrogram with a conventional ventricular sensing electrode. An earlier occurring trigger event may be detected by extracting the His bundle conduction potential from a special ventricular sensing electrode using signal processing techniques.

Although the invention has been described in conjunction with the foregoing specific embodiments, many alternatives, variations, and modifications will be apparent to those of ordinary skill in the art. Other such alternatives, variations, and modifications are intended to fall within the scope of the following appended claims.

What is claimed is:

1. A method comprising:
mounting an anchor member at a surface of a heart, the anchor member having a tension member coupled to the anchor member;
advancing a lead body along the tension member, the lead body including a plurality of electrodes disposed along the lead body;
identifying an MI region of the heart;
positioning the plurality of electrodes at or near the MI region;
affixing the tension member to the lead body to hold the electrodes in position; and
delivering pulses through the plurality of electrodes to the heart.

2. The method of claim 1, identifying the MI region includes identifying the MI region utilizing the plurality of electrodes.

3. The method of claim 1, wherein mounting the anchor at the surface of a heart includes positioning the anchor member adjacent an exterior surface of a myocardium.

4. The method of claim 1, wherein identifying the MI region includes sensing a decrease in R wave amplitude.

5. The method of claim 1, wherein identifying the MI region includes measuring activation-recovery intervals at a plurality of myocardial sites.

6. The method of claim 1, wherein positioning the plurality of electrodes at or near the MI region includes positioning at least one electrode within the MI region and at least one electrode in a peri-MI region.

7. The method of claim 1, wherein delivering pulses through the plurality of electrodes to the heart includes delivering a pacing pulse output sequence selected to pre-excite selected myocardial sites within the MI region.

8. A method comprising:
identifying an MI region of a heart;
inserting an anchor into the heart proximate the MI region, the anchor coupled to a tension member;
positioning the anchor member adjacent a surface of the heart;
advancing a lead body over the tension member such that a plurality of electrodes disposed along the lead are positioned at or near the MI region;
affixing the tension member to the lead body; and
delivering pulses through the plurality of electrodes to the MI region.

9. The method of claim 8, wherein positioning the anchor member adjacent a surface of the heart includes positioning the anchor member adjacent an exterior surface of a myocardium.

10. The method of claim 8, wherein identifying the MI region includes sensing a decrease in R wave amplitude.

11. The method of claim 8, wherein identifying the MI region includes measuring activation-recovery intervals at a plurality of myocardial sites.

12. The method of claim 8, wherein the plurality of electrodes at or near the MI region are positioned such that at least one electrode within the MI region and at least one electrode in a peri-MI region.

13. The method of claim 8, wherein delivering pulses through the plurality of electrodes to the heart includes delivering a pacing pulse output sequence selected to pre-excite selected myocardial sites within the MI region.

14. An apparatus comprising:
a lead including a plurality of electrodes;
an anchor member coupled to a tension member, the lead including a passage with the tension member located within the passage such that the lead is held in position at a heart when the anchor member is at a surface of the heart and the tension member is tautly coupled to the lead body; and
a controller coupled to the plurality of electrodes, the controller adapted to deliver pacing pulses to a pre-identified MI region of the heart via one or more of the plurality of electrodes.

15. The apparatus of claim 14, wherein the controller senses the MI region of the heart via the plurality of electrodes.

16. The apparatus of claim 14, wherein the anchor member is adapted for abutting a surface of the heart, the anchor being inserted through the myocardium to an operating position.

17. The apparatus of claim 14, wherein the tension member includes a stop located at a distance from the anchor for fixing the lead body to prevent movement in either a forward or a rearward direction.

18. The apparatus of claim 14, wherein the apparatus includes circuitry for delivering pacing pulses in a selected pulse output sequence such that the MI region is excited before other myocardial regions.

19. The apparatus of claim 14, wherein the controller senses the MI region by measuring an activation-recovery interval as the time between a detected depolarization and a detected repolarization in an electrogram generated by a sensing channel.

20. The apparatus of claim 14, wherein the controller senses the MI region by sensing a decrease in R wave amplitude.
